(19) United States
(12) Patent Application Publication
(10) Pub. No.: US 2026/0013845 A1
(43) Pub. Date: Jan. 15, 2026

(54) MEDICAL FASTENING ADAPTER WITH QUICK-CHANGE COUPLING FOR DETACHABLE FASTENING TO A NON-STERILE MEDICAL DEVICE

(71) Applicant: B. Braun New Ventures GmbH, Freiburg (DE)

(72) Inventors: Lukas Hahn, Sigmaringen (DE); Malte Kurbjeweit, Aachen (DE); Jan Dahmen, Seitingen-Oberflacht (DE); Thomas Scholten, Tuttlingen (DE)

(21) Appl. No.: 19/260,791

(22) Filed: Jul. 7, 2025

(30) Foreign Application Priority Data
Jul. 10, 2024 (DE) 10 2024 119 621.1

(51) Int. Cl. A61B 17/00; A61B 34/30; A61B 90/00

(52) U.S. Cl. CPC A61B 17/00; A61B 34/30; A61B 90/08; A61B 2017/00486; A61B 2090/0813

(57) ABSTRACT

A medical fastening adapter detachable fastens to a fastening section of a medical robot, which can be arranged within a sterile cover. The adapter has a support structure with an instrument interface and at least a first, second and third geometric coupling structure. Each coupling structure can form a positive connection with the fastening section. An adjuster changes the positions of the first, second, and third geometric coupling structures relative to one another so that, in a first state, the fastening adapter can be coupled to the fastening section, and in a second state, the fastening adapter is non-detachably fixed to the fastening section. The adjuster further fixes a position of the fastening adapter relative to the fastening section via the first, second, and third coupling structures. A medical fastening system can be used to detachably fasten a fastening adapter to a fastening section of a medical robot.

### CROSS-REFERENCE TO RELATED APPLICATION

This application claims priority under 35 U.S.C. § 119 to German Application No. 102024119 621.1, filed on July 10, 2024, the content of which is incorporated by reference herein in its entirety.

### FIELD

The present disclosure relates to a medical fastening adapter with a quick-change coupling (or quick-change mechanism) that is designed and intended for positioning (medical) instruments in a precise position relative to a medical facility or medical device (e.g. a robot or retaining arm, a robot-guided surgical microscope, a stand, a patient bed/chair, etc.) or to guide them along an axis, even if a sensitive sterile barrier is inserted between them, without damaging it.

Such a medical device or facility, which is usually located in a non-sterile region, is typically enclosed by a sterile cover as a sterile barrier, wherein the fastening adapter provides an interface for a medical product in the sterile region, such as a medical, in particular a surgical, instrument. The medical fastening adapter with quick-change coupling preferably comprises a support structure (also referred to as base structure/base frame) with a coupling device for (temporarily/detachably) mounting the adapter to a corresponding fastening section of the medical device and the instrument interface/coupling interface adapted to couple or uncouple (by means of the predefined instrument interface) a sterile medical product, such as an instrument or another medical device. In addition, the present disclosure relates to a medical fastening system and a medical device with a sterile cover for fastening sterile medical products to the medical device.

### BACKGROUND

In surgical procedures, for example, medical robots, in particular medical surgical microscopes, retaining stands and similar medical devices, are typically used in combination with other medical products such as surgical instruments, an additional endoscope or a camera. Accordingly, it is advantageous if the medical devices (robots) can be expanded via a coupling to include the respective medical products, in particular surgical instruments, depending on the desired requirements. To achieve this, it is essential that the sterile instruments do not come into direct contact with the non-sterile medical equipment (robots). A sterile barrier, e.g., in the form of a sterile cover, is usually placed between the instruments to be coupled and the medical robot.

In the state of the art, there are approaches that enable a sterile medical instrument to be fastened to a medical device (robot), e.g., a surgical microscope. Conventionally, intended sterile barriers may be damaged or even destroyed by this fastening maneuver, which is why in normal cases only a single fastening with subsequent single use of the respective sterile barrier is possible. This means that a medical instrument cannot be refastened to the medical non-sterile device (robot) without additional intermediate steps. Furthermore, approaches exist that are intended to allow multiple changes or alignments to the orientation of medical instruments attached to a medical device (robot), but the precision and repeatability of mounting medical instruments over a sterile barrier of these conventional approaches are severely limited.

It is extremely important that the sterile instruments can be coupled to the medical devices (robots) easily and efficiently and that the sterile barrier is not compromised by the coupling process, particularly by repeated coupling maneuvers.

### SUMMARY

In view of this, the present disclosure is based on the task of avoiding or at least mitigating the disadvantages described above and, in particular, providing a medical fastening adapter, a medical fastening system, and a medical device, in particular a medical robot, for the detachable fastening of a sterile medical product, such as an instrument to the medical device such as a robot, with which a stable and precise connection between the components to be coupled (device and product) can be established and which can be handled as easily as possible. Furthermore, a sub-task can be seen in designing the fastening adapter or fastening system in such a way that a sterile barrier, in particular in the form of a sterile (plastic) cover, arranged between the medical device and the sterile product (instrument) to be coupled remains intact or is not damaged by repeated coupling operations.

The present disclosure therefore provides for the provision of a fastening adapter with a mounting frame (hereinafter also referred to as support structure or base frame) with at least two, preferably at least three (arranged or formed at an angle to each other on the mounting frame) geometric coupling/engagement structures for engaging with a medical (non-sterile) device. At least one of the coupling/engagement structures is designed or equipped with a (manually operated) adjustment mechanism which is designed and constructed for this purpose, to change (increase and decrease) the distance between the engagement positions/engagement locations/engagement points formed/defined by the coupling/engagement structures and the medical device, and also to clamp the medical device between the coupling/engagement structures of the fastening adapter. In other words, the at least one adjustment mechanism is designed or intended to change and/or preload the engagement position/engagement location/engagement point of the coupling/engagement structure provided with it relative to the engagement positions/engagement locations/engagement points of the other coupling/engagement structures. According to the disclosure, at least one adjustment mechanism is preferably equipped with a limiter that is designed and configured to limit the contact/clamping force applied to the medical device by the associated coupling/engagement structure to a predetermined or predeterminable value. At least one coupling/engagement structure, preferably one that has (includes) the adjustment mechanism, is additionally provided with or operatively connected to a manually operated quick-release device (quick-change coupling), by means of which the coupling/engagement structure in question can be quickly removed from (at least partially bypassing, in particular without actuating) the adjustment mechanism and any limiter, can be quickly removed from the fastening section of the medical device and preferably quickly returned to the fastening section of the medical device, wherein the previously set and, if applicable, limited contact force via the coupling/engagement structure and/or the limiter is preferably automatically or, preferably, by means of (minor) readjustment by means of the adjustment mechanism.

In other words, the present disclosure relates to a medical fastening adapter for detachable fastening to a fastening section of a medical device, for example a robot, in particular a robot-guided surgical microscope, wherein the medical device, in particular the medical robot, is preferably arranged within a sterile cover as a sterile barrier, and wherein the medical fastening adapter has a (rigid/dimensionally stable) support structure (as a kind of basic framework) with an instrument interface. The medical fastening adapter also has at least one first geometric coupling structure, a second geometric coupling structure, and preferably a third geometric coupling structure, each of which is adapted and designed for a form-fitting and/or friction-locking connection to the fastening section, and an adjustment/fixing/locking mechanism which is adapted to change the relative spatial position of the at least first, second and preferably third geometric coupling structures relative to one another in such a way that, in a first state of a first relative positioning, the fastening adapter can be coupled to and uncoupled from the fastening section and, in a second state of a second relative positioning, it is permanently fixed/fastened/locked to the fastening section, and by means of the first, second and preferably third coupling structures, a defined/unique position of the fastening adapter relative to the fastening section is determined. For example, the first, second, and preferably third coupling structures can point radially inward toward the fastening section and be further apart from each other in a first relative position than in the second relative position, wherein in the first relative position there is still a distance/clearance (i.e., still without rigid contact) between the coupling structures and the fastening section, so that the fastening adapter is freely movable and can be aligned or arranged with respect to the fastening section, and in the second relative position, the coupling structures are moved relative to each other, thereby reducing the distance between them and establishing geometric contact with corresponding fixation as a result.

In other words, the fastening adapter has at least two, preferably at least three geometric coupling structures/elements (for unambiguous determination of a position in which a plane can be defined by three points) which are designed and constructed to couple/mount/fasten the fastening adapter to a fastening section which is formed or arranged on the medical device/robot. The coupling structures are preferably arranged on a common level on the support structure. The fastening adapter (for fixing and releasing) has an adjustment mechanism (adjustment means/adjustment element or fixing means) which, when actuated, allows the relative position of the geometric coupling structures to be changed/influenced, in particular manually.

The function of the adjustment mechanism can be described again as follows: Actuating the adjustment mechanism moves the fastening adapter from a first state of a first relative positioning of the geometric coupling structures with respect to each other, in which the fastening adapter can be coupled to the fastening section of the robot or uncoupled from the fastening section of the robot, and into a second state of a second relative positioning of the geometric coupling structures relative to each other, in which the fastening adapter is fixedly secured to the fastening section. This means that in this second state, the coupling structures of the fastening adapter are aligned with each other in such a way that the fastening adapter is fastened to the fastening section (by form and/or friction) via these coupling structures, in particular spans or clamps around it, so that it is fixed to the fastening section of the robot without the adjustment means having to be actuated again to release it (i.e., the position of the fastening adapter on the fastening section is fixed). Thus, in this second state, a clear position of the fastening adapter relative to the fastening section of the robot is defined or there are no degrees of freedom for a relative movement of these two coupled components to each other.

In order to maintain a predetermined or predeterminable clamping force which can be applied by the coupling structures to the fastening section of the medical device (maximum), the adjustment mechanism disclosed is preferably equipped with a limiter (e.g., slip coupling) which is functionally arranged between the relevant coupling structure (e.g., clamping arm, clamping finger, clamping bolt, etc.) and a handle (e.g., rotary knob, tension lever, etc.) for adjusting the position of the coupling/engagement structure, and by means of which this clamping force can be prevented from being exceeded. This makes it possible to apply a maximum load to a sterile barrier inserted between the fastening adapter (the coupling structures) and the medical device (its fastening section), for example in the form of a film, using only the predetermined or predeterminable clamping force, which is selected or selectable accordingly so that damage to the sterile barrier can be prevented or avoided.

The fastening adapter is also provided with a manually operable quick-release device which is designed and constructed so that, when actuated, at least one of the coupling structures, preferably the coupling structure which has the adjustment mechanism, from the fastening section of the medical device and preferably quickly returning it to the previous clamping position with the fastening section of the medical device without the (manual) handle and any limiter connected in series having to be actuated. This ensures that the clamping force previously set via the limiter is automatically restored.

Preferably, the quick-release device is arranged functionally parallel to the adjustment mechanism (adjustment means), in particular parallel to the limiter and to the handle of the relevant coupling structure (integrated into the adjustment mechanism). Alternatively, the quick-release device can also be arranged/formed on another of the coupling structures that does not have an adjustment mechanism.

Preferably, the medical fastening adapter has a sterile-side (instrument) interface to which the medical product, in particular a sterile product such as an instrument, that is ultimately to be fastened can be mounted/connected. The fastening adapter thus serves as a (serial) intermediate link for the (sterile) medical product and the (non-sterile) medical device/robot, with the film sandwiched between the coupling structures and the fastening section acting as a sterile barrier.

Advantageous embodiments are explained in particular below.

In a first advantageous embodiment, the first coupling structure, the second coupling structure, and the third coupling structure lie in a plane and each have a protruding/projecting convex surface, in particular as a hemisphere, in order to engage positively, for example as a locking lug, in a complementary counter-coupling structure (on the fastening section of the medical device). At least the convex surface of the first coupling structure faces the convex surfaces of the second and/or third coupling structures, and the convex surface of the first coupling structure can be translated/axially displaced by means of the adjustment mechanism (hereinafter also referred to as the adjustment means) in order to change the relative distance to the second and/or third coupling structure. The convex surface, particularly in the form of the hemisphere, therefore provides a rounded coupling structure without sharp edges or points, which cannot damage a sterile cover if it is clamped between the fastening adapter and the fastening structure. In other words, the fastening adapter has three coupling structures, each of which is arranged in a common plane and each of which has a convex / curved / partially spherical geometry protruding from the support structure, in particular in the form of a hemisphere. The coupling structures are arranged relative to one another in such a way that a first of the coupling structures or its protruding geometry faces the second and/or the third coupling structure. Preferably, the second and third coupling structures point in the same direction and are arranged parallel to each other. Furthermore, the first coupling structure is axially displaceable by manual actuation of the adjustment means with which the first coupling structure is connected, namely in an axial relative movement towards or away from the second and third coupling structures, whereby the axial relative distance between the first coupling structure and the remaining coupling structures can be changed. This type of design with convex, in particular hemispherical, coupling structures allows the fastening adapter to be aligned with a high degree of precision and at the same time blocks the degrees of freedom of the fastening adapter relative to the medical robot in a coupled state. Advantageously, such a curved design of the coupling structures as contact points does not have any pointed or angular geometries, which could potentially damage/break through a sterile cover/barrier arranged between the contact points of the fastening adapter with the medical robot.

In a further preferred embodiment of the disclosure, the second coupling structure and the third coupling structure, in particular with their convex surfaces, are rigidly arranged on or opposite the support structure, in particular formed in one piece in the support structure.

The adjustment device is preferably designed as a rotatable screw gear with a rotary handle or rotary knob for manual operation, which, when rotated, adjusts the position of the first coupling structure, preferably with the convex surface, relative to the support structure and thus also relative to the second and third coupling structures. In other words, the relative position of the first coupling structure, in particular the convex surface, can be adjusted by rotating the rotary handle, which is arranged on the adjustment means. In particular, the first coupling structure is engaged with the adjustment means via a threaded connection, as a result of which the coupling structure can be displaced or is displaced axially through the thread by a rotary movement of the rotary handle. This design with the screw gear and the operable rotary handle ensures the most precise fine adjustment possible. In particular, such a thread compensates for tolerances that may arise due to the overlying sterile cover. The support structure of this fastening adapter in this embodiment is designed, for example, in the form of a fork with a second and third coupling structure, which are each arranged on the fork arms or alternatively, in a further embodiment, has a closed (peripheral) contour, on the inner peripheral contour of which, in particular on one side, the second and third coupling structure are arranged. The support structure of this alternative design with a closed contour is formed in such a way that it encompasses the entire periphery of a fastening section.

According to a further preferred embodiment, the screw gear has a limiter/torque limiter (which is rigidly connected to the rotary handle and) which, when a limit torque is exceeded, only transmits the limit torque and also provides the operator with haptic and/or acoustic feedback. In other words, an (additional) torque limiter, which is preferably located inside the screw gear, limits the torque that is applied to the rotary handle by the operator. Preferably, if a limit torque is exceeded, a spinning of the screw gear is initiated, whereby no further screwing process and therefore no further translational movement of the first coupling structure is possible. This prevents the adjustment means from being overtightened, which could lead to damage to the sterile barrier or the fastening adapter and/or fastening section components themselves. Alternatively or additionally, the torque limiter enables a haptic and/or acoustic signal to be emitted to the operator so that the operator recognizes when a limit torque has been reached. For example, when the limit torque is exceeded, a click can be emitted as an acoustic signal as well as a haptic signal, similar to a torque wrench.

In particular, the fastening adapter can be designed and constructed as a reusable component, for example for sterilization in an autoclave.

According to a further embodiment, the support structure of the fastening adapter can have a guide stop (as a type of limiting edge) in the form of a projection, which is formed in the region of the first coupling structure and in particular projects in the same direction as the first coupling structure and lies parallel to the plane of the first second and third coupling structure, in order to geometrically align the fastening adapter in the first state of the first relative positioning with respect to a fastening section by means of the guide stop. Such a guide stop ensures that the fastening adapter and in particular the first coupling structure can be guided and arranged in a correct position or alignment to the fastening section during the coupling process. This prevents incorrect, especially skewed, alignment of the fastening adapter.

In a further advantageous embodiment of the disclosure, the quick-release device provided in addition to the adjustment means is designed as follows in the case of an arrangement integrated into the adjustment means according to the above structure:

The internal threaded sleeve described above, preferably equipped with the limiter, is mounted in an outer sleeve so that it can rotate and move axially. A peripheral clearance is formed between the two sleeves, into which a spring (coil spring) is inserted, which surrounds the internal threaded sleeve on the outside. This spring is supported at one axial end against a first stop/spring seat, which is connected in a rotatable (but axially fixed) manner to the internal threaded sleeve, and at its other axial end against a second stop/spring seat, which is connected/formed in a fixed/one- piece manner to the outer sleeve, such that the internal threaded sleeve is preloaded axially in the direction of engagement of the coupling structure (push bolt) mounted therein relative to the outer sleeve. This coil spring arrangement allows complete axial retraction of the rotary handle and the internal threaded sleeve axially fixed (but relatively rotatable) to it and the coupling structure (push bolt) screwed into it with respect to the outer sleeve without the rotary handle having to be rotated.

Alternatively, however, the quick-release device provided in addition to the adjustment means may also be provided on a coupling structure different from the coupling structure comprising the adjustment means. In this case, the quick-release device may be designed according to a buckle principle with a tension lever and pull buckle or pull strap and preferably have the following construction:

Preferably, the first, second, and third coupling structures are preferably located in one plane, the support structure has a first arm with a first coupling structure and a second arm with a second coupling structure, and the first coupling structure is located opposite the second coupling structure, and the first and/or second arms are designed such that they can be elastically deflected relative to each other to enable relative positioning of the first coupling structure relative to the second coupling structure.

In this preferred design, the quick-release device provided in addition to the adjustment means has a hinge-like lever pull/snap lever with a form-fitting/tensionable (bendable but not stretchable in the longitudinal direction due to the low thickness of the steel strip) steel strip, in particular further comprising two clamping jaws on the steel strip, which connect the two arms to each other and, together with the arms, form a closed peripheral contour, and a shortening of the steel strip between two points can be adjusted via the hinge-like lever pull, which preferably results in an elastic deformation of the first and second arms towards each other, in order to reduce a relative distance between the first coupling structure and the second coupling structure and to fix the fastening adapter in the second state of the second relative positioning to the fastening section. In other words, the fastening adapter preferably has two arms that are preferably flexible/elastic relative to each other, which can be moved relative to each other within a predetermined range by applying force and each have a lateral coupling structure, in particular in the form of a curved projection. The arms are fastened with a flexible steel strip / steel plate, which is arranged on the outer contour of the fastening adapter and connects the two arms at the front and which can be shortened using a hinge-like lever as an adjustment means. The shortening or contraction of the steel strip also requires, in particular, the pressing of sliding jaw elements, which are further applied to the fastening interface by actuating the lever. This narrows the periphery of the fastening adapter, which causes the adapter to grip tightly around the fastening section of the medical robot and places the fastening adapter in the second state of the second relative positioning of the coupling structures. The fastening adapter has a third coupling structure, which is arranged in particular on the inner contour side of the fastening adapter opposite the steel strip, in particular with the clamping jaws, and which has the adjustment means, if necessary with a limiter.

According to a further embodiment, the fastening adapter has the instrument interface for the connection/mounting of a sterile medical product, in particular an instrument, as well as two alignment elements or projections which are arranged on the inner contour side which has the third coupling structure and are intended to prevent lateral displacement of the fastening adapter during a mounting process.

In a further preferred aspect of the disclosure, the fastening adapter, in particular the support structure, has a (high-performance) plastic, preferably PA 66 GRP or PEEK GRP, as material, in particular the support structure as a whole is made of this material. This ensures that no electrical contact is made between the fastening adapter and the medical robot. In particular, the support structure of the fastening adapter can be made of a mechanically resilient or stronger material, in particular metal as a material, preferably stainless steel or a titanium alloy, and the electrical insulation can be realized via a (separate) insulation sleeve (preferably made of PEEK), in particular at the front instrument interface.

The present disclosure further relates to a medical fastening system for detachable fastening to a fastening section of a medical device (robot), if necessary with the insertion of a sterile barrier/sterile cover (film) between the medical fastening system and the medical device, in particular a robot-guided surgical microscope, which is arranged in particular within the sterile cover as a sterile barrier. The fastening system has a separate fastening adapter as disclosed, and the fastening section has at least a first, second, and preferably third (counter-) coupling structure, which are (each) formed in a form-fitting and/or force-fitting manner and are complementary to or the first coupling structure, the second coupling structure and preferably the third coupling structure of the fastening adapter and cooperate with each other. In other words, the fastening section, with which the fastening adapter can be coupled, has corresponding (complementary) counter-coupling structures which are (geometrically) designed in such a way that the respective coupling structures of the fastening adapter engage geometrically and positively in them during the coupling process. The coupling structures and counter-coupling structures of the fastening adapter and fastening section are therefore specially matched to each other.

In particular, according to an aspect of the present disclosure, a set comprising a first fastening adapter according to the present disclosure and an associated first fastening section, as well as a different second fastening adapter according to the present disclosure with an associated second fastening section, which are not interchangeable with one another, may be provided, wherein the first and second fastening sections are mounted or can be mounted on the medical device (robot) so that only the first fastening adapter can be coupled to the first fastening section and only the second fastening adapter can be coupled to the second fastening section in order to prevent confusion by medical personnel. In this way, a set can be provided that enables a defined local assignment of the fastening adapter (to the associated complementary fastening section) without accidental replacement.

In a (particularly first) advantageous embodiment, the fastening section of the medical fastening system has a plate-shaped base structure with a central opening perpendicular to the plane of the base structure (for example to enable a surgical microscope to be viewed through this opening) and, on a front side (i.e., a side surface of the plate-shaped base structure), a first counter- coupling structure with a concave notch, which is aligned in the front direction and whose alignment lies in the plane of the base structure, and has, on a side region lateral to the central opening, a second and a third counter-coupling structure with a concave notch, which are each aligned in the opposite direction to the front direction, so that in the case of the fastening adapter with the first, the second and the third coupling structure, which lie in one plane and each have a projecting/protruding convex surface, this convex surface interacts in each case with the concave notch in order to fix a position of the fastening adapter relative to the fastening section by means of positive locking of the at least three contact points. In other words, a central (through) opening is arranged in the plate- shaped base structure of the fastening section, in which the medical device (robot), in particular the surgical microscope, can be arranged. The fastening section has three concave counter-coupling structures that are designed in such a way that they accommodate the respective coupling structures of the fastening adapter in a complementary manner during a coupling process.

In a further (in particular second) advantageous embodiment, the fastening section of the medical fastening system has a plate-shaped base structure with a central opening perpendicular to the plane of the base structure (or the longitudinal axis of the opening is perpendicular to the plate- shaped base structure) and, on a front side, a first counter-coupling structure with a concave notch, which is aligned in the front direction (longitudinal axis of the concave notch is aligned in the front direction) and lies in the plane of the base structure, and, on a rear side, which is arranged opposite the front side of the base structure, a second and a third counter-coupling structure, each with a concave notch, which are each aligned opposite the front direction, so that in the case of the fastening adapter with the first, the second, and the third coupling structure, which lie in one plane and each have a protruding/projecting convex surface, this convex surface interacts in each case with the concave notch in order to fix a position of the fastening adapter relative to the fastening section by means of positive locking (via the relative movement by the adjustment means) of the at least three contact points.

In particular, the second and third counter-coupling structures are arranged next to each other on the rear side of the base structure, which is opposite the front side with the first counter-coupling structure. In other words, in this embodiment, all three counter-coupling structures are arranged on the base structure in such a way that the coupling structures of the fastening adapter of this embodiment, which are arranged along the closed inner contour of the fastening adapter, can engage or grip into it in a complementary manner.

In the further (in particular third) advantageous embodiment, the fastening section has a plate-shaped base structure with a central opening perpendicular to the plane of the base structure and has a first counter-coupling structure and a second counter-coupling structure arranged on two opposite side walls and a third counter-coupling structure on a front side, so that in the fastening adapter with the first arm with the first coupling structure and the second arm with the second coupling structure, which are designed such that they can be deflected elastically relative to one another, these first, second, and third coupling structures interact with the respective counter- coupling structure, in particular in interaction with the two clamping jaws on the steel strip which connects the two arms to one another, in order to fix a position of the fastening adapter relative to the fastening section by means of positive locking of the at least three contact points.

In a further advantageous aspect of the disclosure, the medical fastening system comprises a separate sterile cover arranged or arrangeable between the fastening adapter and the fastening section and in contact with at least the first, second, and third counter-coupling structure of the fastening section and the first, second, and third coupling structure of the fastening adapter. In other words, a sterile cover/sterile barrier, in particular in the form of a sterile plastic film/cover, is arranged between the sterile fastening adapter and the (non-sterile) fastening section of the medical robot, whereby the sterility of the medical instrument to be coupled is not impaired/influenced by the mounting/coupling to the medical device (robot) via the fastening adapter. In particular, the medical fastening system is designed in such a way that the sterile cover can be easily positioned or clamped between the coupling structures of the fastening adapter and the complementary counter- coupling structures of the fastening section without any risk of damage or breaking through the sterile barrier. In particular, the fastening adapter only has rounded surfaces and (round) "edges" on the coupling structures, which engage in the counter-coupling structures indirectly via the sterile cover, so as not to damage the sterile cover.

The present disclosure further relates to a medical device, in particular a medical robot, for example in the form of a robot-guided surgical microscope, with a sterile cover for fastening / coupling sterile medical products to the medical device. The medical device, in particular the robot, has at least one fastening section and at least one fastening adapter according to the disclosure, in particular a plurality of fastening sections and fastening adapters, for example as a fastening adapter set, or the medical device, in particular the robot, has a fastening system according to the disclosure (with fastening section and fastening adapter according to the present disclosure). The sterile cover is arranged between the fastening section and the fastening adapter fastened to it in such a way that the robot with the fastening section(s) is/are arranged in a non-sterile region and the fastening adapter with, for example, a medical instrument is arranged in a sterile region and thus separated from each other

Any disclosure related to the fastening adapter of the present disclosure also applies to the fastening system of the present disclosure as well as vice versa.

### BRIEF DESCRIPTION OF THE DRAWINGS

The disclosure is explained in more detail below with reference to advantageous embodiments and with reference to the associated figures.

FIG. 1 shows a perspective (top) view of a partial section of a medical fastening system according to a first advantageous embodiment of the disclosure;

FIG. 2 shows in a plan view of a sectional view through a plane of the medical fastening system according to the first advantageous embodiment of the disclosure, which is spanned by the three coupling structures of the fastening adapter;

FIG. 3 shows a perspective view of the medical fastening system according to the first advantageous embodiment in a state coupled to a surgical microscope and thus also a medical device in the form of a robot according to a preferred embodiment;

FIG. 4 shows a perspective view of a fastening adapter according to a first advantageous embodiment;

FIG. 5 shows a perspective front view of a fastening section according to a first advantageous embodiment of the disclosure for a medical robot;

FIG. 6 shows a further perspective rear view of the fastening section according to the first advantageous embodiment of the disclosure;

FIG. 7 shows a longitudinal side view through a partial section of the fastening adapter according to the first embodiment in the coupled state with the fastening section according to the first embodiment of the disclosure;

FIG. 8 shows a longitudinal sectional side view through the adjustment means of the fastening adapter in a decoupled state of the fastening adapter;

FIG. 9 shows another longitudinal sectional side view through the adjustment means of the fastening adapter, in a coupled state of the fastening adapter;

FIG. 9A shows a longitudinal sectional side view through the adjustment means of the fastening adapter according to the disclosure,

FIG. 9B shows a perspective view of a retaining sleeve of the adjustment means according to FIG. 9A for securing a coupling structure against rotation, preferably in the form of a push-screw bolt.

FIG. 10 shows a perspective (side) view of a medical fastening system according to a second advantageous embodiment of the disclosure;

FIG. 11 shows a perspective view of the medical fastening adapter according to a second advantageous embodiment of the disclosure;

FIG. 12 shows a perspective longitudinal sectional view through the adjustment means of the fastening adapter in a coupled state of the fastening adapter;

FIG. 13 shows a perspective longitudinal sectional side view through one of the second or third coupling structures of the fastening adapter according to the second embodiment of the disclosure, in a coupled state of the fastening adapter;

FIG. 14 shows a perspective side view of the medical fastening system according to the second advantageous embodiment in a state coupled with a surgical microscope;

FIG. 15 shows a longitudinal sectional side view through the adjustment means/fastener in an alternative embodiment;

FIG. 16 shows a perspective top view of a medical fastening system according to a third advantageous embodiment of the disclosure;

FIG. 17 shows a perspective view of the medical fastening adapter according to a third

advantageous embodiment of the disclosure with a quick-release device provided separately from the adjustment means not shown;

FIG. 18 shows a further perspective view of the medical fastening adapter according to the third advantageous embodiment of the disclosure;

FIG. 19 shows a perspective (front) view of the fastening section according to a third advantageous embodiment of the disclosure;

FIG. 20 shows a perspective (side) view of the fastening section according to a third advantageous embodiment of the disclosure;

FIG. 21 shows a perspective side view of the medical fastening system according to the third embodiment during a coupling process; and

FIG. 22 shows a longitudinal side view through an alignment element of the fastening adapter according to the third embodiment during a coupling operation.

The figures are merely schematic in nature and are provided solely for the purpose of understanding the present disclosure. It should be noted that the features of the individual embodiments are interchangeable and may occur in a particular combination.

### DETAILED DESCRIPTION

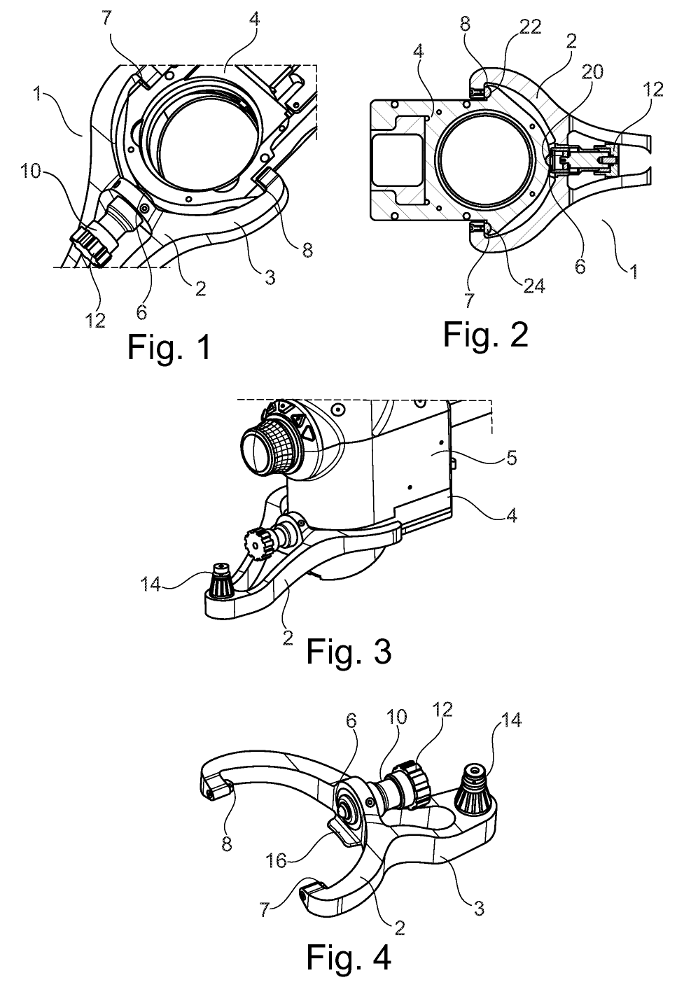

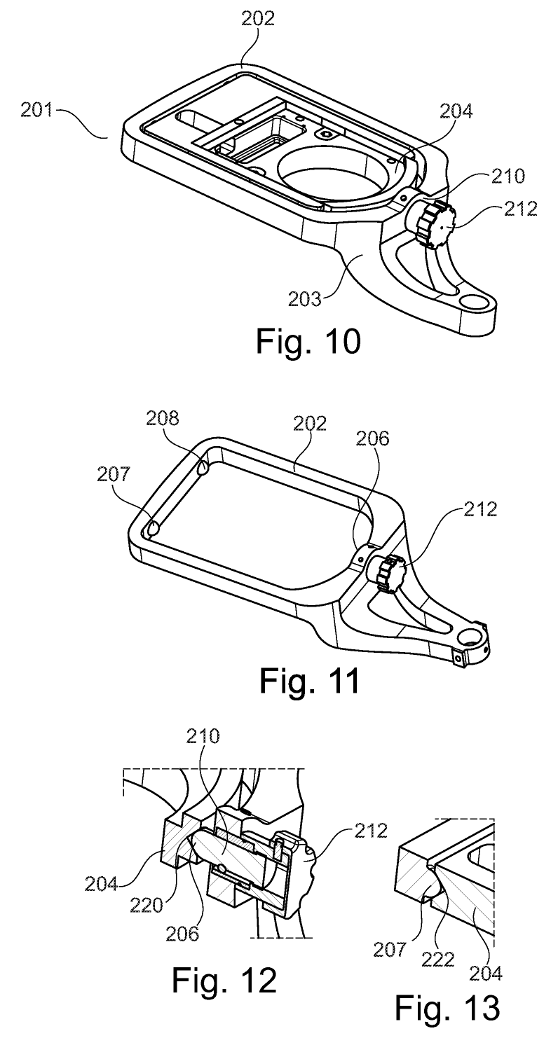

In the following, the present disclosure is described with reference to the advantageous embodiments with reference to FIGS. 1 through 22. FIGS. 1 through 9 show views of a fastening system according to a first preferred embodiment comprising a fastening adapter of a first preferred embodiment and an (associated) fastening section of a first preferred embodiment for a medical device, in particular medical robot according to a preferred embodiment. FIGS. 10 through 15 further show a fastening system according to a further, second preferred embodiment with a fastening adapter and a fastening section, each according to a second preferred embodiment. FIGS. 16 through 22 finally show a fastening system according to a further, third preferred embodiment with a fastening adapter and a fastening section each according to a third preferred embodiment.

FIG. 1 shows a perspective view of a partial section of a fastening system 1 and FIG. 2 asectional view through a plane which is spanned by three coupling structures 6, 7, 8 of a fastening adapter 2, according to the first advantageous embodiment of the medical fastening system 1. The fastening adapter 2 according to the first advantageous embodiment has a support structure 3, in particular a fork-shaped structure. The fastening adapter 2 is shown in a coupled state with a (separate) fastening section 4, which is designed as part of a medical robot 5 or can be connected to it. The coupling or contact between the components of the fastening adapter 2 and the fastening section 4 takes place via a first coupling structure 6, a second coupling structure 7 and a third coupling structure 8 of the fastening adapter 2, which in this first embodiment are designed as convex / curved surfaces in the form of spherical hemispheres. These three coupling structures 6, 7, 8 are arranged in one plane and engage in three complementary counter-structures 20, 22, 24 of the fastening section 4, which in this first embodiment are designed as (complementary) concave indentations (for example with recessed hemispherical surfaces). The first coupling structure 6 is arranged on a central axis of the fastening adapter 2 and is directed against the second coupling structure 7 and the third coupling structure 8. It can also be said that the first coupling structure 6 with the convex surface is opposite the second and third coupling structures, and in this embodiment a longitudinal axis of the first coupling structure 6 is parallel to the longitudinal axes of the second and third coupling structures 7, 8, wherein the directions of the convex surfaces are opposite.

The second and third coupling structures 7, 8 are arranged at the end sections of the fork-like arms of the support structure 3 of the fastening adapter 2, in particular along an imaginary common axis, and are at the same time laterally spaced from the first coupling structure 6. Furthermore, the fastening adapter 2 has an adjustment means 10 in the form of a screw gear, which can be operated manually via a rotary handle 12. This adjustment means 10 is in connection with the first coupling structure, whereby the first coupling structure 6 can be displaced translationally / axially along the central axis of the fastening adapter in the direction of the second and third coupling structures 7, 8 or away from them when the rotary handle 12 is turned manually. In this way, the fastening adapter can be transferred from a first state of a first relative positioning of the coupling structures 6, 7, 8 relative to one another to a second state of a second geometric relative positioning by means of the adjustment means 10, as also explained in detail below.

FIG. 3 shows a perspective side view of the medical fastening system 1 according to the first advantageous embodiment in a state coupled with a robot-guided surgical microscope 5. FIG. 3 thus also shows the medical robot according to a preferred embodiment. The fastening section 4 can be designed as an integral part of the surgical microscope 5 or alternatively, as in the present case, can be mounted as a separate component of a fastening section 4 in an additional step on this robot- guided surgical microscope 5. FIG. 3 also shows a cylindrical or conical instrument interface 14 for a sterile medical instrument. In the coupled state of the fastening adapter 2, the instrument interface 14 is perpendicular to a plane of the fastening system 1 or a plane of the fastening adapter (i.e., parallel to an axis of a central opening) and is arranged on the end section of the support structure 3 which faces the fork-shaped arms of the support structure 3. The support structure 3 can also be described as Y-shaped with two opposing arms, wherein the instrument interface 14 is arranged on the section facing away from the arms (the lower part of the Y, so to speak). In a side view, the support structure 3 also does not lie entirely in one plane, but the arms with the second and third coupling structures 7, 8 and the adjustment means 10 with the first coupling structure 6 lie essentially in one plane, which is adjoined by a sloping section with a distal front section with the instrument interface 14, wherein the front section is again offset parallel to the plane of the arms (in the manner of a step in order to ensure accessibility to the adjustment means 10).

FIG. 4 shows a perspective view of the fastening adapter 2 according to the first advantageous embodiment. As described above, the support structure 3 of the fastening adapter of this first embodiment has a fork-like shape with two arms. One of the second and third coupling structures 7, 8 is arranged at the end section of each of these arms. The first coupling structure 6, which is connected to the adjustment means 10, is arranged in the middle of the support structure 3. The adjustment means 10 has a rotary handle 12, by means of which the first coupling structure 6 can be axially displaced (rotation is converted into translation). One end section of the support structure 3 has a conical (instrument) interface 14 for the coupling of a medical instrument as a medical product. In addition, the support structure 3 has a projecting guide stop 16 below the first coupling structure 6, which is formed in one piece with the support structure 3 and is intended to geometrically align the fastening adapter 2 with respect to the fastening section 4. The guide stop 16 protrudes in the same direction as the first coupling structure 6.

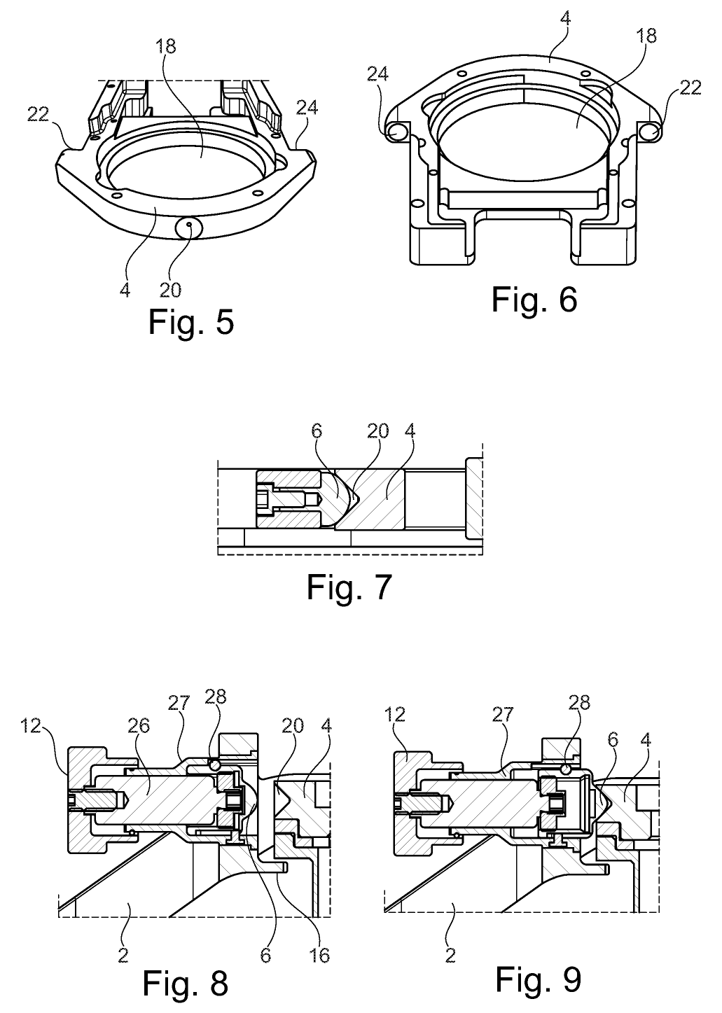

FIGS. 5 shows a perspective front view of a fastening section 4 and FIG. 6 shows a perspective rear view of the fastening section 4 according to a first advantageous embodiment of the disclosure for a medical robot.

The fastening section 4 according to this first embodiment has a plate-shaped base structure with a central opening 18 perpendicular to the plane of the base structure. The opening 18 is intended in particular to accommodate a lens of a medical surgical microscope 5 (centrally). On its front side, the base structure has a first counter-coupling structure 20 with a concave notch that is aligned in the front direction and lies in the plane of the base structure. The two averted side walls of the base structure each have a projection in the lateral direction, on which the second and third counter-coupling structures 22, 24 are each integrated with a concave notch, which are each aligned in the opposite direction to the first counter-coupling structure 20 (their virtual longitudinal axes, starting from the notch, point away from the first counter-coupling structure). The first, second, and third counter-coupling structures 20, 22, 24 lie in the same plane and are designed and adapted to receive the coupling structures 6, 7, 8 of the fastening adapter 2 in a complementary manner in order to interact with them and to fix a position of the fastening adapter 2 relative to the fastening section 4 by means of positive locking of the at least three contact points in the second state of the second relative positioning.

FIG. 7 shows a longitudinal sectional side view through a partial section of the fastening adapter 2 according to the first embodiment in the coupled state with the fastening section 4 according to the first embodiment of the disclosure. In this view, the hemispherical first coupling structure 6 of the fastening adapter 2, which is firmly connected to the adjustment means 10 not shown here, preferably via a screw connection, engages in the counter-coupling structure 20 of the fastening section 4 provided for this purpose. FIG. 7 thus shows a coupled state of the fastening adapter 2 and the fastening section 4.

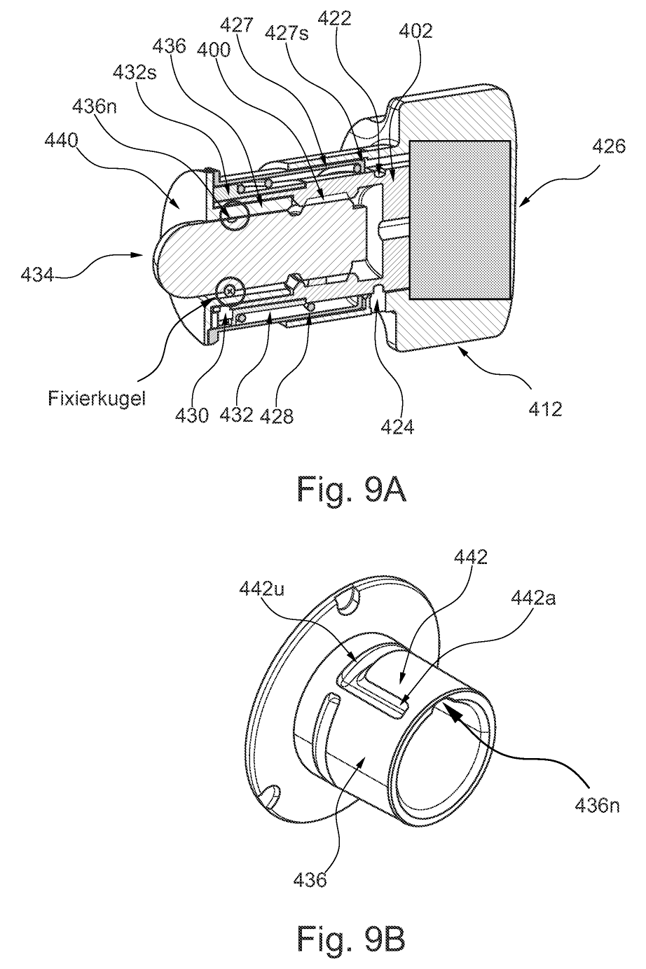

FIG. 8 shows a longitudinal sectional side view through the adjustment means 10 of the fastening adapter 2 in its basic configuration, in a first decoupled state of the fastening adapter 2, and FIG. 9 shows the same longitudinal sectional side view in a second coupled state of the fastening adapter 2. Accordingly, the adjustment means 10 is generally designed in the form of a screw gear with a rotary handle 12. By manually turning the rotary handle 12, the first hemispherical coupling structure 6 is moved axially in the direction of the first counter-coupling structure 20 provided on the fastening section 4 and ultimately brought into positive (active) engagement. The adjustment means 10 of the fastening adapter 2 preferably has a torque limiter 26 which ensures that only the limit torque is transmitted when a limit torque is exceeded. The limiter also gives the operator a haptic and/or acoustic feedback signal. Furthermore, a fixing ball 28 is arranged in an eccentric axial groove within a shell/sleeve 27 of the screw thread, and the fixing ball 28 simultaneously engages in a spherical notch/milling on the outer periphery of the first coupling structure 6. This prevents rotation of the first coupling structure 6 together with the rotary handle 12. Thus, the fixing ball 28 moves together with the first coupling structure 6 in the direction of the counter-coupling structure 20 when the rotary handle 12 is actuated for a coupling operation of the fastening adapter 2. A purely axial displacement of the first coupling structure 6 ensures that an existing sterile cover, which is clamped between the first coupling structure 6 and the first counter-coupling structure 20, is wound up or destroyed.

FIG. 9A shows an adjustment means/adjustment mechanism with integrated quick-release device in accordance with a preferred exemplary embodiment of the disclosure in detail.

Accordingly, the adjustment means has a hollow rotary handle or rotary knob 412, inside which a limiter/torque limiter 426 is accommodated. The rotary handle 412 is also mounted on an outer sleeve 427 so that it can rotate and move axially, with an internal threaded sleeve 400 being mounted in the outer sleeve 427 so that it can rotate and move axially. The outer sleeve 427 also serves as a mounting sleeve for securely mounting the entire adjustment means (including the coupling structure) in/on the support structure 3 of the fastening adapter.

The internal threaded sleeve 400 also protrudes axially from the outer sleeve 427 in the direction of the rotary handle 412 and is coupled to the rotary handle 412 via the limiter 426 for torque transmission. For this purpose, the internal threaded sleeve 400 has an engagement device arranged inside the rotary handle 412, for example in the form of a friction element, a locking element or a similar force transmission component (not shown in detail), whereas the rotary handle 412 is provided with an engagement means, for example in the form of a spring-preloaded or preloadable counter-friction element, engagement balls or pins, locking teeth or similar components (not shown in further detail), which can be brought into disengageable operative engagement with the engagement device. In principle, such limiters 426 are well known in the prior art, for example in the form of slip clutches (see, for example, Wikipedia), so that reference can be made here to the relevant disclosures.

Furthermore, the internal thread sleeve 400 and the rotary handle 412 are axially fixed. For this purpose, a circumferential groove 422 is formed on the jacket side of the internal threaded sleeve 400, into which a grub screw 424 engages radially in a sliding manner, which is screwed radially into the rotary handle 412. This grub screw 424 thus holds the internal threaded sleeve 400 axially in the rotary handle 412, but allows relative rotation of the rotary handle 412 with respect to the internal threaded sleeve 400. This means that the rotational movement of the rotary handle 412 is transmitted to the internal threaded sleeve 400 exclusively via the limiters 426, wherein the limiter 426 is triggered when a specific/determinable torque is exceeded, thus interrupting the torque transmission between the rotary handle 412 and the internal threaded sleeve 400.

A peripheral clearance is formed between the internal threaded sleeve 400 and the outer sleeve 427, into which a (quick-release) preload spring 428, preferably in the form of a coil spring, is inserted. The coil spring 428 is supported by a spring seat 427s formed by the outer sleeve 427 close to the axis of the rotary handle 412 (one-piece) and by a spring seat 432s mounted axially fixed on the internal threaded sleeve 400 axially distant from the rotary handle, thereby pre- tensioning the internal threaded sleeve 400 in the direction away from the rotary handle 412. This means that the rotary handle 412 is pulled/pressed axially against the outer sleeve 427 by the coil spring 428 via the internal threaded sleeve 400. To form the axially spaced spring seat 432s, a push screw bolt 430 is screwed into the internal threaded sleeve 400, by means of which a spring seat sleeve 432 is screwed to the jacket side of the internal threaded sleeve 400, which forms the axially removed spring seat 432s (in one piece).

The coupling structure, preferably in the form of a push(-in) bolt 434, is screwed into the internal threaded sleeve 400. To prevent the push bolt 434 from rotating within the internal threaded sleeve 400, a retaining sleeve 436 is provided, which is inserted in an axial section of the internal threaded sleeve 400 between the latter and the push bolt 434 and has an internal longitudinal groove 436n in which a radial projection on the push bolt side (retaining/fixing ball shown only schematically in FIG. 9B) is guided axially in a sliding manner. The retaining sleeve 436 also has a face plate 440, which is firmly connected to the outer sleeve 427 and is therefore fixed in rotation by the latter.

To quickly release the coupling structure (push bolt) 434 from the fastening section of the medical device (robot), it is therefore not necessary to turn the actuating knob 412 (multiple times), but simply pulled back against the preload force of the coil spring 428 relative to the outer sleeve 427 (which also serves as a mounting sleeve) in order to also pull back the internal threaded sleeve 400 and the push bolt 434 screwed into it. When the rotary knob 412 is released, the coil spring 428 pushes the internal threaded sleeve 400 and the push bolt 434 screwed into it back into their original position relative to the outer sleeve 427, from which they were retracted.

Finally, on the jacket side of the retaining sleeve 436, a guide frame 442, for example in the form of a bayonet lock, is preferably formed, into which a radial projection 444 on the internal threaded sleeve side slides. The guide frame 442 has, as shown in FIG. 9B, an axially extending section 442a, which preferably merges into a peripherally extending section 442u.

The functionality of this design can be summarized as follows:

As long as the radial projection 444 on the side of the internal threaded sleeve is located in the axially extending section 442a of the guide frame 442, the rotary handle 412 together with the internal threaded sleeve 400 and the push bolt 434 can be easily pulled back or released against the coil spring 428, in the latter case, the coil spring 428 displacing the internal threaded sleeve 400 and the push bolt 434 within the (stationary) outer sleeve 427 until the radial projection 444 on the internal threaded sleeve side comes into contact with the peripherally extending section 442u of the guide frame 442. At this point, the rotary handle 412 must be actuated and a rotary movement of the internal threaded sleeve 400 must be effected via the limiter (slip coupling) 426, whereby the push bolt 434 is displaced axially out of the internal threaded sleeve 400 until a maximum contact force is exerted on the fastening section of the medical device (with the sterile barrier between them) and the limiter 46 is triggered (the slip coupling slips). In this case, the contact pressure is transferred to the outer sleeve 427 via the guide frame 442 and the radial projection 444 on the internal threaded sleeve side, wherein the coil spring 428 remains unloaded. This has the advantage that the coil spring 428 can be designed with a comparatively low spring stiffness, whereas without the guide frame described above, the contact pressure would be exerted via the coil spring, which would therefore have to have a high spring stiffness. The latter would make it possible to manually retract the rotary handle, but would make this more difficult.

FIG. 10 shows a perspective view of the medical fastening system 201 according to a second advantageous embodiment of the disclosure. The fastening adapter 202 in this second embodiment has a support structure 203 with a closed contour (in contrast to the two arms as in the first embodiment). This support structure 203 completely surrounds the outer contour of the fastening section 204 of this second embodiment. The fastening adapter 202 of this second embodiment also has a fastener 210 with a rotary handle 212 similar or identical to the fastener 10 shown in FIG. 9A.

FIG. 11 shows a perspective view of the medical fastening adapter 210 according to the second advantageous embodiment of the disclosure. The support structure 203 of the fastening adapter 202 has a closed contour for enclosing the outer contour of a fastening section 204. The first coupling structure 206 is connected to the adjustment means 210, which can be actuated via the rotary handle 212, and points into the interior of the closed contour of the support structure. The second coupling structure 207 and the third coupling structure 208 are arranged on the inner side, which is opposite the side with the first coupling structure 206. An imaginary axis of the first coupling structure 206 extends through the center axis of the fastening adapter 202 and the second and third coupling structures 207, 208 are arranged on imaginary axes which are each spaced at an equal distance, parallel, from the imaginary axis of the first coupling structure 206. The first coupling structure 206 also points in the opposite direction to that in which the second and third coupling structures 207, 208 point, and thus towards each other.

FIG. 12 shows a perspective longitudinal side view through the adjustment means 210 of the fastening adapter 202 according to the second advantageous embodiment in a coupled state of the fastening adapter 210, and FIG. 13 shows a perspective longitudinal side view through one of the second or third coupling structure 207, 208 of the fastening adapter 202 according to this second embodiment in a coupled state of the fastening adapter 202. The adjustment means/fastener 210 of this second embodiment is in particular also designed as the fastener shown in FIG. 9A. FIG. 12 shows the engagement of the first coupling structure 206, which is rigidly connected to the fastener 210, with the complementary counter-coupling structure 220 of the fastening section 204. FIG. 13 shows the positive engagement of the second coupling structure 207 with the complementary counter-coupling structure 222 of the fastening section 204.

FIG. 14 shows a perspective side view of the medical fastening system 201 according to the second advantageous embodiment in a state coupled to a robot-guided surgical microscope 5 (as a medical robot according to the present disclosure). The support structure 203 of the fastening adapter 202 completely embraces a fastening section 204, which is arranged in a section of the surgical microscope 5. The sterile cover preferably arranged between the surgical microscope 5, in particular the fastening section 204, and the fastening adapter 202 is not shown in FIG. 14.

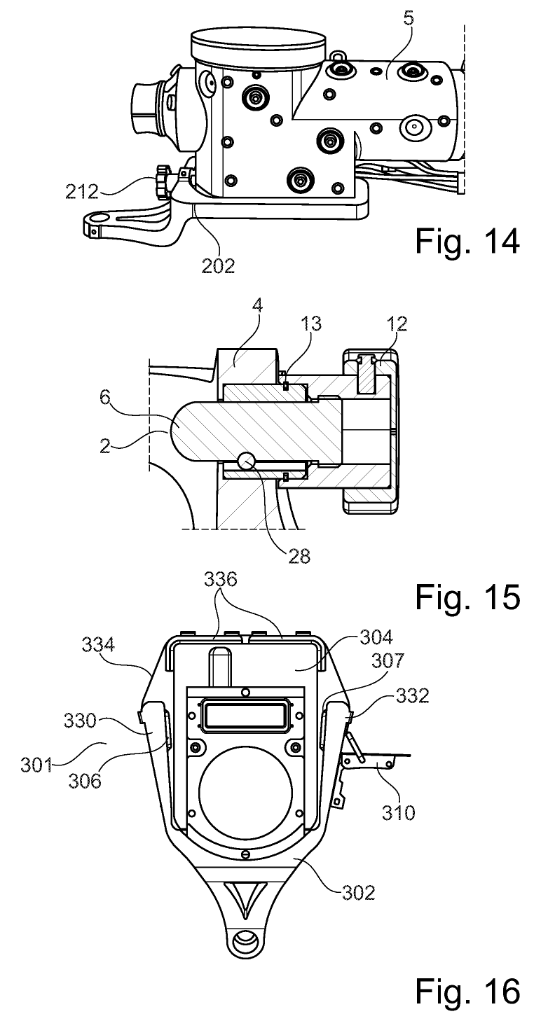

FIG. 15 shows a longitudinal side view through an alternative embodiment of the fastener 10 for the first (or the second) embodiment of the medical fastening adapter 2. This alternative embodiment of the fastener 10 does not have a torque limiter 26. The sleeves of the fastener 10 are connected here via a snap ring 13. Furthermore, this design also has the fixing ball 28, which prevents the first coupling structure 6 from twisting. The quick-change device is not shown in detail.

FIG. 16 shows a perspective top view of the medical fastening system 301 according to a third advantageous embodiment of the disclosure. The fastening adapter 302 in this third embodiment has a first arm 330 and a second arm 332 arranged opposite it, which are elastically formed. In this illustration, the fastening adapter 302 is already aligned with the fastening section 304, but is not yet coupled. The coupling sometimes takes place via an engagement of the first coupling structure 306 and the second coupling structure 307 in the respective counter-coupling structures 322, 324 (not shown here) of the fastening section 304. Furthermore, the fastening adapter 302 is additionally retained by two clamping jaws 336, which are connected to a flexible steel strip 334 which can also be shortened. These clamping jaws 336 are adapted to the outer contour of the fastening section 304. The fastener itself is not shown, but the additional quick-release device in this embodiment has a hinge-like lever pull 310 (still in a first coupled and uncoupled state in FIG. 16). Actuation of the lever pull 310 causes the first and second resilient arms 330, 332 and thus also the first coupling structure 306 and the second coupling structure 307 to deflect relative to one another and causes the steel strip 334 to shorten, thereby fixing a position of the fastening adapter 302 relative to the fastening section 304 or coupling the two components. The lever pull 310 fastened to one arm thus causes a contraction of the steel strip fastened to the other, opposite arm with accompanying contraction and elastic deflection of the arms towards each other, whereby a relative distance between the first and second coupling structure is shortened (i.e., from a first relative positioning towards a second relative positioning).

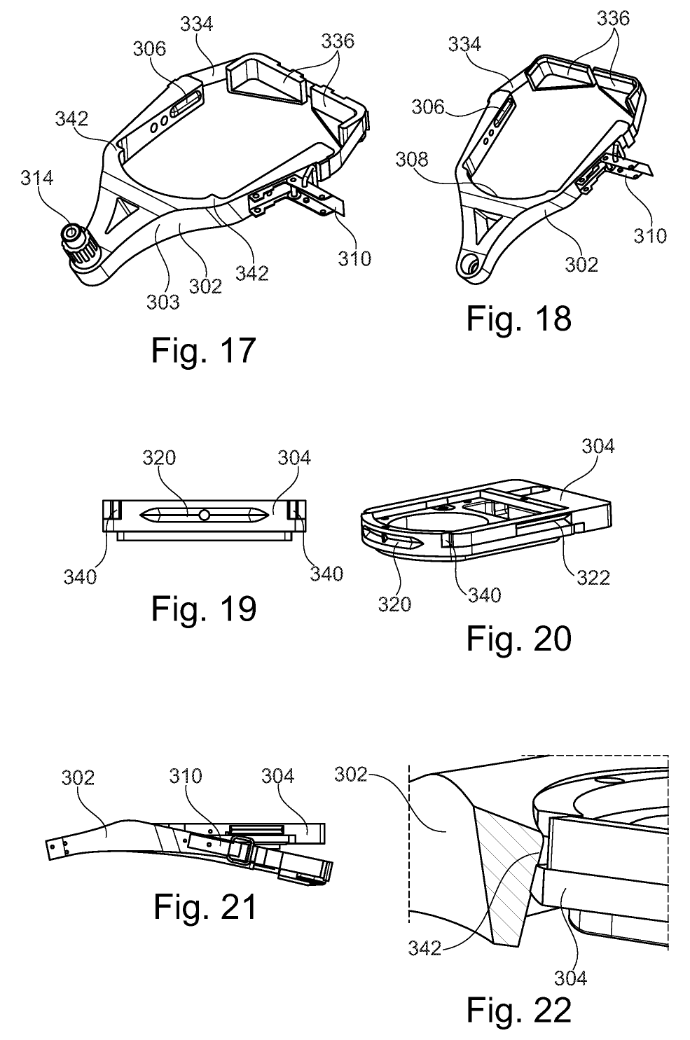

FIG. 17 and FIG. 18 each show a perspective side view of the medical fastening adapter 302 according to the third advantageous embodiment of the disclosure. The support structure 303 of the fastening adapter 302 has a conical/cylindrical instrument point 314 at one end section for the connection of a medical instrument (not shown in FIG. 18). Furthermore, the support structure 303 of the fastening adapter 302 has a third coupling structure 308 (not shown here) on an inner side, which is arranged opposite the clamping jaws 336. The first, second, and third coupling structures 306, 307, 308 are preferably shaped as elongated, in particular stadium-shaped, protrusions. Furthermore, the support structure 303 has two projection-like alignment elements 342, which are arranged on the inner contour side and lie opposite the clamping jaws 336 as viewed in the longitudinal direction. These alignment elements 342 are designed to prevent lateral displacement of the fastening adapter 302 during a mounting / coupling operation to the fastening section 304.

FIG. 19 shows a perspective front view and FIG. 20 a perspective side view of the fastening section 304 according to a third advantageous embodiment of the disclosure. The third counter- coupling structure 320 is arranged on the front side of the fastening section 304 and has an elongate, in particular stadium-shaped, notch as a geometry into which the third coupling structure 308 of the fastening adapter 302 can engage in a complementary manner. The first and second counter- coupling structures 322, 324 are arranged on the side walls of the fastening section 304, which preferably form a rectangular recess on the side walls. In addition, indentations 340 are provided at the respective corners of the fastening section 304, which are arranged between the side walls and the front side, each of which receives an alignment element 342 of the fastening adapter 302 in a complementary manner during a coupling process.

FIG. 21 shows a perspective side view of the medical fastening system 301 and FIG. 22 shows a longitudinal side view through an alignment element 342 of the fastening adapter 302 according to the third embodiment during a coupling process. It can be seen from FIG. 21 that a coupling or mounting process of the fastening adapter 302 to the fastening section 304 first begins with an alignment of the third coupling structure 308 with the associated counter-coupling structure 320 and the two alignment elements 342 with the associated indentations 340 on the fastening section 304. The opposite end section of the fastening adapter 302, on which the clamping jaws 336 are arranged, is angled with respect to the fastening section 304 and is pulled over it at an angle, so to speak. This is possible because the steel strip 334 with the clamping jaws 336 is in the loosened or extended state, whereby in this state the inner periphery of the fastening adapter 302 is larger than the outer periphery of the fastening section 304. Only after the lever pull 310 is actuated does the steel strip 334 with the clamping jaws 336 shorten and at the same time the first arm 330 and the second arm 332 are deflected towards each other, whereby the fastening adapter 302 is coupled to the fastening section 304.

1. A medical fastening adapter for detachably fastening or guiding a medical instrument to a fastening section of a medical device, the medical fastening adapter comprising:
a support structure with an instrument interface;
at least a first geometric coupling structure and a second geometric coupling structure, each of which is designed for a form-fitting and/or friction-locking connection to the fastening section;
an adjuster adapted to change a relative position of the first geometric coupling structure and the second geometric coupling structure relative to one another in such a way that in a first state of a first relative positioning, the medical fastening adapter is configured to be coupled to and uncoupled from the fastening section, and in a second state of a second relative positioning, the medical fastening adapter is fixedly secured to the fastening section; and
a quick-release device that is manually operable and configured to quickly remove at least one of the first geometric coupling structure and the second geometric coupling structure from the fastening section of the medical device, at least partially bypassing the adjuster.

2. The medical fastening adapter according to claim 1, wherein the quick-release device is integrated into the adjuster.

3. The medical fastening adapter according to claim 1, wherein the adjuster is a screw gear.

4. The medical fastening adapter according to claim 3, wherein the screw gear has a torque limiter which is operatively connected to a rotary handle and which, when a limit torque is exceeded, only transmits the limit torque.

5. The medical fastening adapter according to claim 1, wherein the adjuster comprises:
a hollow actuating handle which is rotatably and axially displaceably mounted on an outer mounting sleeve;
an internal threaded sleeve which is rotatably and axially displaceably received in the outer mounting sleeve and is retained axially fixed in the hollow actuating handle in such a way that torque can be transmitted from the hollow actuating handle to the internal threaded sleeve; one of the first geometric coupling structure and the second geometric coupling structure screwed into the internal threaded sleeve and retained in such a way that, when the internal threaded sleeve is rotated, said one of the first geometric coupling structure and the second geometric coupling structure is displaced axially; and a preload spring that axially preloads the internal threaded sleeve relative to the outer mounting sleeve in a direction of the second relative positioning and allows axial displacement of the internal threaded sleeve relative to the outer mounting sleeve.

6. The medical fastening adapter according to claim 5, wherein:
the outer mounting sleeve forms a first spring seat for the preload spring, and
a bearing sleeve is fixed on the internal threaded sleeve on a jacket side, the bearing sleeve forming a second spring seat for the preload spring.

7. The medical fastening adapter according to claim 5, further comprising a retaining sleeve which is firmly connected to the outer mounting sleeve, wherein said one of the first geometric coupling structure and the second geometric coupling structure is mounted in the retaining sleeve in a rotationally fixed and axially displaceable manner in axial sections.

8. The medical fastening adapter according to claim 7, wherein:
the retaining sleeve has a shell side,
the retaining sleeve has or forms a guide frame on the shell side with a first guide section extending exclusively in an axial direction and a second guide section extending in a peripheral direction,
the internal threaded sleeve has a projection protruding radially inward,
the projection engaging in the guide frame in a sliding manner,
. first guide section allowing the internal threaded sleeve to be withdrawn, and
the second guide section allowing the internal threaded sleeve to be rotated through a predetermined angle of rotation corresponding to a length of the second guide section.

9. The medical fastening adapter according to claim 1, further comprising a third geometric coupling structure.

10. The medical fastening adapter according to claim 9, wherein:
the first geometric coupling structure, the second geometric coupling structure, and the third geometric coupling structure lie in one plane;
each of the first geometric coupling structure, the second geometric coupling structure, and the third geometric coupling structure having a convex surface,
at least the convex surface of the first geometric coupling structure faces the convex surface of the second geometric coupling structure and/or the third geometric coupling structure, and
the convex surface of the first geometric coupling structure is displaceable in translation by the adjuster to change a relative distance.

11. The medical fastening adapter according to claim 9, wherein the second geometric coupling structure and the third geometric coupling structure are rigidly arranged on the support structure.

12. The medical fastening adapter according to claim 11, wherein the second geometric coupling structure and the third geometric coupling structure are formed in one piece in the support structure.

13. The medical fastening adapter according to claim 9, wherein the support structure has a guide stop comprising a projection, the projection formed in a region of the first geometric coupling structure and projects in a same direction as the first geometric coupling structure and lies parallel to a plane of the first geometric coupling structure, the second geometric coupling structure, and the third geometric coupling structure, in order to geometrically align the medical fastening adapter with respect to the fastening section by the guide stop in the first state of the first relative positioning.

14. The medical fastening adapter according to claim 9, wherein:
the first geometric coupling structure, the second geometric coupling structure and the third geometric coupling structure lie in one plane, the support structure has a first arm with the first geometric coupling structure and a second arm with the second geometric coupling structure, the first geometric coupling structure lies opposite the second geometric coupling structure, the first arm and the second arm are elastically deflectable relative to one another, the quick-release device comprises a hinge-like lever pull with a form-fitting steel strip that connects the first arm to the second arm, and a shortening of the steel strip between two points is adjustable via the hinge-like lever pull, which results in an elastic deformation of the first arm and the second arm towards each other to reduce a relative distance between the first geometric coupling structure to the second geometric coupling structure and to fix the medical fastening adapter to the fastening section in the second state of the second relative positioning.

15. A medical fastening system comprising the medical fastening adapter according to claim 1, wherein:
the medical fastening adapter further comprises a third geometric coupling structure,
the medical fastening system being configured to detachably fasten the medical fastening adapter to a fastening section of a medical robot, and
the first geometric coupling structure, the second geometric coupling structure and the third geometric coupling structure configured to interact positively and complementarily with a first counter-coupling structure, a second counter-coupling structure and a third counter-coupling structure of the fastening section of the medical robot.

16. The medical fastening system according to claim 15, wherein the fastening section has a plate-shaped base structure with a central opening perpendicular to a plane of the base structure and has, on a front side, a first counter-coupling structure with a concave notch which is aligned in a front direction and, on a side region lateral to the central opening, a second and a third counter-coupling structure, each with a concave notch, which are each aligned in an opposite direction to the front direction, so that when the medical fastening adapter with the first geometric coupling structure, the second geometric coupling structure and the third geometric coupling
structure, which lie in one plane and each have a convex surface, this convex surface interacts in each case with the concave notch in order to fix a position of the medical fastening adapter relative to the fastening section by positive locking of at least three contact points.

17. The medical fastening system according to claim 15, wherein the fastening section has a plate-shaped base structure with a central opening perpendicular to a plane of the base structure and has a first counter-coupling structure with a concave notch on a front side, which is aligned in a front direction, and on a rear side, which is arranged opposite the front side of the base structure, has a second and a third counter-coupling structure, each with a concave notch, which are each aligned in an opposite direction to the front direction, so that when the medical fastening adapter with the first geometric coupling structure, the second geometric coupling structure, and the third geometric coupling structure, which lie in one plane and each have a convex surface, this convex surface interacts in each case with the concave notch in order to fix a position of the medical fastening adapter relative to the fastening section by positive locking of at least three contact points.

18. The medical fastening system according to claim 15, wherein the fastening section has a plate-shaped base structure with a central opening perpendicular to a plane of the base structure and has a first counter-coupling structure and a second counter-coupling structure arranged on two remote side walls and a front side has a third counter-coupling structure, so that in the medical fastening adapter with a first arm with the first geometric coupling structure and a second arm with the second geometric coupling structure, which are designed in such a way that they can be deflected elastically relative to one another, the first geometric coupling structure, the second geometric coupling structure, and the third geometric coupling structure interact with the respective counter-coupling structure in order to fix a position of the medical fastening adapter relative to the fastening section by positive locking of at least three contact points.

19. The medical fastening system according to claim 18, further comprising a separate sterile cover which is arranged between the medical fastening adapter and the fastening section and is connected to at least the first counter-coupling structure, the second counter-coupling structure, and the third counter-coupling structure of the fastening section as well as the first geometric
coupling structure, the second geometric coupling structure, and the third geometric coupling structure of the medical fastening adapter.

20. A medical device comprising:
at least one fastening section;
at least one fastening adapter; and
a sterile cover,
the sterile cover arranged between the at least one fastening section and the at least one fastening adapter so that the medical device is arranged in a non-sterile region, and
the at least one fastening adapter arranged with approximately one medical instrument is arranged in a sterile region.
